(19) United States
(12) Patent Application Publication
(10) Pub. No.: US 2003/0114377 A1
(43) Pub. Date: Jun. 19, 2003

(54) Inhibition therapy for septic shock with mutant CD14

(76) Inventors: Theo N. Kirkland, La Jolla, CA (US); Suganya Viriyakosol, San Diego, CA (US)

Correspondence Address: BROWN, MARTIN, HALLER & MCCLAIN LLP, 1660 UNION STREET, SAN DIEGO, CA, 92101-2926, US

(21) Appl. No.: 10/251,718

(22) Filed: Sep. 20, 2002

Related U.S. Application Data
(63) Continuation of application No. 09/443,638, filed on Nov. 18, 1999.
(60) Provisional application No. 60/109,227, filed on Nov. 18, 1998.

(51) Int. Cl. A61K 38/17; C07K 14/74; C12P 21/02; C12N 5/06

(52) U.S. Cl. 514/12; 530/350; 435/69.1; 435/320.1; 435/325

(57) ABSTRACT

Described are methods and novel therapeutics for treating CD14-mediated diseases, especially sepsis and septic shock. The methods and compounds are founded on principles of structural mimicry of the innate compound. Changes to the innate compound are described that result in unexpected properties useful for blocking or ameliorating the harmful effects of systemic infection by microbes. Diagnostic applications and product-by-process claims are also contemplated based on the same underlying principle and observation.

### CROSS-REFERENCE TO RELATED APPLICATIONS

This application claims priority to U.S. Provisional Application Ser. No. 60/109,227 filed Nov. 18, 1998, which is herein incorporated by reference in its entirety including drawings.

### STATEMENT REGARDING FEDERALLY SPONSORED RESEARCH

This work was supported by the Public Health Service, grant P01 GM37696, and by the Medical Research Service of the Department of Veterans Affairs. The U.S. government may have certain rights in the invention.

### FIELD OF THE INVENTION

The field of invention relates to protein and genetic engineering with the objective of achieving new, pharmacologically active compounds and therapeutic methodologies based on structural mimicry of the naturally occurring biological compound, CD14.

### BACKGROUND OF THE INVENTION

Septic shock remains a major problem in clinical medicine. It is estimated that 200,000 cases of septic shock occur each year in the

U.S. alone, with a mortality rate approaching 50% (1,2). Septic shock remains the most common cause of death in intensive care units (1,2).

The syndrome of septic shock is caused by over production of a host of inflammatory cytokines by monocytes and macrophages in response to microbial products. These include tumor necrosis factor (TNF)¹, IL-1, IL-6, IL-8 [reviewed in (3,4)]. Macrophages also secrete a wide variety of other compounds in response to microbial products, including platelet activating factor, prostaglandins, enzymes, and reactive oxygen intermediates (4). Endothelial cells respond directly to lipopolysaccharide (LPS), as well as to cytokines secreted by macrophages (5-7). The expression of tissue factor by endothelial cells can activate the clotting cascade (8) and endothelial cell production of nitric oxide may play a role in vasodilatation (9). Production of all these mediators can culminate in hypotension, intravascular coagulation, poor organ perfusion, multi-organ failure and ultimately, death (1).

A major breakthrough in our understanding of the molecular mechanisms of septic shock has been the realization that CD14 is a receptor for both LPS and peptidoglycan (10-12), two of the most abundant constituents in the bacterial cell wall. We will use the term “LPS receptor” to indicate the ability of CD14 to facilitate the activation of cells by LPS. CD14 functions both as a cell membrane receptor and a soluble receptor for bacterial LPS (5-7,10). CD14 is expressed as a glycophosphatidylinositol-linked protein on the surface of macrophages, monocytes and polymorphonuclear phagocytes (13) and many laboratories have shown that CD14 is a critical part of the LPS recognition system in those cells (10, 11, 14, 15). Soluble CD14 (sCD14) also plays a crucial role in the LPS response of endothelial and epithelial cells (5-7). It is noteworthy that sCD14 did not function as an effective soluble receptor for peptidoglycan despite the fact that sCD14 bound peptidoglycan very well (16).

CD14 has also been reported to be a membrane receptor for a very wide range of ligands. The bacterial products include lipoarabinomanan (17), lipoetichoic acid (18), a uronic acid polymer (19), spirochete lipoproteins (20) and a surface protein on the pathogenic fungus Blastomyces dermatitidis (21). CD14 has also been reported to bind to apoptotic cells (22). Both sCD14 and membrane CD14 have been reported to bind to phospholipids (23,24). This wide spectrum of reactivity fits the definition of a “pattern recognition receptor”, as proposed by Janeway (25). Thus, CD14 seems to be an important part of the innate immune system. The structural basis for this wide spectrum of activity is not known.

We have previously characterized the LPS receptor activity of a group of deletion mutants of CD14 expressed as membrane proteins on Chinese Hamster Ovary cells (26) and the mouse pre-B cell, 70Z/3 (27,28). In this report, we evaluate the activity of this same group of CD14 mutants as soluble LPS receptors, and report that they have different activities than when expressed as membrane receptors. This implies that soluble and membrane forms of CD14 are functionally distinct in their roles as LPS receptors.

Because of the widespread myriad effects of the CD14 receptor, a greater understanding of its structure and function is needed, particularly as leads to the treatment of CD14-mediated diseases such as sepsis and septic shock. New insight is provided by this disclosure, with favorable implications for diagnosis and treatment.

### SUMMARY OF THE INVENTION

In a first aspect, the invention features a modified mammalian CD14 receptor comprising: a) a first domain capable of binding a membrane component from a foreign body with an enhanced affinity relative to an unmodified mammalian CD14 receptor, and b) a second domain or region distinct from the first that, when physically altered or disrupted, reduces the transmission of a normal CD14-associated cellular signal.

In one embodiment, the modified mammalian CD14 receptor is substantially full-length relative to the corresponding unmodified receptor.

In another embodiment, the binding domain of the modified mammalian CD14 receptor is effective to bind membrane components (e.g., LPS or peptidoglycans) of gram negative bacteria, gram positive bacteria, fungi, protozoa or other microbes implicated in the etiology of sepsis or septic shock in a mammalian host, preferably a human.

In preferred embodiments, the enhanced affinity of the binding domain is a consequence of the alteration of the second domain, which alteration may occur at a distance of preferably about 50 linear amino acid residues. Lesser and greater distances are also contemplated.

In particularly preferred embodiments, the alterations are mutations of specific amino acid residues, e.g., residues located at approximate positions 7-12 and 22-25 of Seq ID. No.1. In a most preferred embodiment, each of the above are removed from the second domain of the human CD14 receptor (Seq ID. No. 1) to result in a substantially full-length CD14 receptor having a binding affinity for LPS that is about 40-fold greater (>1 order of magnitude) than the wild-type CD14 analog but substantially lacking the ability to transmit a normal cellular signal, e.g., in a normal or sepsis-afflicted mammalian host. In other embodiments, the modification is a mutation or series of mutations to the same domain, including amino acid insertions and changes to specific amino acid residues or sequences of residues such as described above. The mutational changes may be “conservative” changes as known in the art, or else non-conservative changes, e.g., replacement of polar residues with non-polar residues. Preservation of the reading frame is intended for embodiments in which the modified receptor is supplied in nucleic acid format. The core invention is a mammalian CD14 receptor that has an increased binding affinity for natural ligand, e.g., LPS, that is mediated by changes made at a remote location, i.e., domain, on the same receptor relative to the first domain. The structural changes, while positively influencing binding affinity at the one domain, are actually physically made to a second domain implicated in another function, i.e., e.g., transmission of a cellular signal. Thus, the manipulation of one domain exerts an effect on another.

In preferred embodiments, the exerted effect is enhanced binding of bacterial membrane components, e.g., LPS and peptidoglycan molecules, that is mediated by physical manipulation of a remote locus or situs on the receptor. This gives rise to other aspects of the invention that will be clear to one of skill in the art, and more apparent from the discussion below.

A second aspect to the invention integrates the first aspect into a pharmaceutical composition that is effective to quell, ameliorate, or reverse adverse symptoms associated with CD14-mediated diseases, e.g., sepsis and septic shock. In particularly preferred embodiments, the pharmaceutical compositions of the invention comprise as the key ingredient the modified CD14 receptor discussed above. This may be administered as either a purified protein, or else as a nucleic acid capable of being expressed, preferably in transient or controlled fashion so as to minimize side effects.

Many possible avenues exist for administration, all of ready determination by one of skill in the art. For example, because functional CD14 receptor serves useful roles in mammalian physiology, it may be desired to anticipate and allow for degradation of the modified receptor once it has effectively taken its course in a host mammalian body afflicted with sepsis. Chronic production of modified CD14, e.g., in the case of permanently engineered or replicating genetic entities, can be disadvantageous following achievement of the desired result, e.g., curing sepsis. Thus, administration of the raw protein (e.g., recombinant, synthesized, and/or purified native from cell culture) or a genetic construct that does not provide for self-replication can be advisable, particularly as a means of controlling the amount and duration of modified CD14 for optimal and transient effect in a patient.

Related to the above, further embodiments make use of further manipulations to the CD14 receptor, e.g., attachment of a targeting molecule, agent, or tag to the receptor in such way as not to negatively effect LPS binding. The tag may serve various functions, i.e., facilitating protein purification and/or allowing for a more rapid clearance of offending infectious agents and microbes, as well as promoting the transient effect and presence of the modified CD14 receptor that is introduced. Other tags may be labels that permit one of skill to “trace” physiological, histological, pathological, and/or therapeutic effects of the compound in vivo or in vitro.

Because normal CD14-mediated signal transmission is desired attenuated according to the invention, it is permissible that such a tag, agent or targeting molecule could simultaneously negate or contribute to the reduction of signaling function. However, in preferred embodiments such an entity is positioned apart from these functional domains, preferably in the COOH terminal half of the full-length or substantially full-length CD14 molecule, insofar as this region is known to be dispensable to LPS binding and cellular signal transmission.

The purpose of a tag moiety, as stated and inter alia, can be to accelerate bacterial clearance, or at least to expedite and/or enhance reversal or amelioration of symptoms associated with sepsis. In preferred embodiments, an immunoglobulin Fc fragment, preferably native to the host recipient mammalian species, e.g., human, may be substituted anywhere in the COOH half of the CD14 receptor, or even added to the full-length or substantially full-length receptor. The probable effect is at least three-fold. First, Fc complexes can form multimers which result in a greater theoretical affinity for antigen or ligand (here microbe components) affinity than the sum of the individual consituent monomers. Second, Fc complexes are targeted by the immune system for clearance, e.g., by macrophages. Third, this component has a great affinity for protein A and G, which are readily conjugated to column and other solid-phase support structures to facilitate purification of the Fc moiety and all to which it is attached.

In a related aspect that builds on the first, a diagnostic kit is contemplated that can assess the relative amount and/or type of receptor substrate present in a given experimental sample, e.g., cell culture homogenate, serum, exudate, or whole blood. This can be accomplished in a variety of formats, as the skilled artisan is aware. In one preferred embodiment, the modified receptor is linked to a solid support medium such as a column, preferably as not to disrupt ligand binding, e.g., LPS. Aqueous experimental sample is then passed there through to identify the presence and/or concentration of the desired ligand. The signaling can be accomplished in a variety of ways, most preferably by a fluor quenching/enhancing system or by an ELISA-based assay. Such procedures are readily familiar to one of skill in the art and the general procedures are further articulated below. Standards, both positive and negative, are also contemplated to allow comparison and validation of experimental results. In other embodiments, a solid support, e.g., an agarose plate may be employed, but serve as a medium on which one or more of the individual or complexed reagents can diffuse or migrate, and interact, e.g., in Ouchterlony plate format. In addition to the above kit formats, corresponding diagnostic methods are also contemplated.

A further aspect is a method of identifying new drugs and/or diagnostic tools that takes cognizance of the unexpected properties that resulted from the experimentation described herein. The method features: a) providing a native receptor that possesses multiple functional domains, e.g., a substrate LPS binding domain and a separate domain associated with cellular signal transmission (e.g., in the case of mammalian CD14 receptor) and b) manipulating either or both domains, followed by c) assaying the specific functionality of the domain not so manipulated. The results described herein prove that this can lead to the fruitful identification of novel, useful compounds. In certain preferred embodiments, the receptor is a mammalian CD14 receptor, preferably murine or human, that is implicated in sepsis or other disease states. In other preferred embodiments, the physical manipulation of one domain intentionally and/or predictably attenuates or enhances the function of that very domain, while simultaneously causing a shift in function of a neighboring domain on the same molecule. This shift in function of the neighboring domain is what is to be assayed according to the method. Because many enzymes are multi-functional and have multiple domains, each with predictable activity and function, this method has great merit in the design of new drugs that mimic natural compounds in structure, but with modulated functionality. The method is readily exploited by one of skill in the art, and can make use of other commonly understood and utilized technologies such as bioinformatics and molecular modeling computer programs. The method can further employ rational drug design as that term of art is commonly used in the field.

A final aspect of the invention is in the manufacture of the modified CD14 receptor. A product-by-process is thus contemplated. It was found, after much toil and cost, that use of a baculovirus expression system results in an unobvious advantage in the amount of recombinant, mutant, functional protein produced. Thus, although expression in bacteria resulted in a substantial yield of recombinant protein, the majority of the protein produced did not possess sufficient biological activity, whereas large quantities of biologically active recombinant protein were obtained from use of the baculovirus system describe herein. This difference did not owe to differences in glycosylation patterns (data not shown), and would appear to be a consequence of a more desirable internal environment that facilitates proper recombinant protein folding. Moreover, multiple other transfected eukaryotic cell lines failed to produce the desired level of protein. Hence, a significant commercial advantage in production costs and other resources is realized by use of the baculovirus system described.

An insect cell line such as described herein, that bears the desired CD14 recombinant gene and that is capable of generating inclusion bodies giving high yields of expressed and functional CD14 within, is therefore also contemplated as another aspect to the invention.

The invention will be better understood from the detailed description of preferred embodiments, taken in conjunction with the accompanying drawings and claims, below.

### BRIEF DESCRIPTION OF THE DRAWINGS

FIG. 1 (corresponding to Seq ID No:1) shows a schematic of soluble CD14 showing the deletion mutants in bold struck through type. The position of glycine 152 corresponding to the last amino acid required for a functional sCD14 and mCD14 receptor (28,35) is shown with a slash. The sequence derived from the vector in the C-terminus is underlined.

FIG. 2 shows an ELISA assay for CD14 binding to Re LPS. The relative amount of LPS binding (compared to wild-type CD14) is shown as a function of increasing amount of LPS. The mean and standard deviation of three determinations is shown; this is one of four representative experiments. Open circles, DDED (residues 9-12 of Seq. ID No: 1; closed circles, PQPD (residues 22-25 of Seq. ID No: 1); open triangles, DDED/PQPD (residues 9-12 and 22-25 of Seq. ID No: 1); closed triangles, DPRQY (residues 59-63 of Seq. ID No: 1); open squares, AVEVE (residues 35-39 of Seq. ID No: 1).

FIG. 3 shows the fluorescence intensity of FITC-LPS (10 ng/ml) interacting with the indicated concentrations of sCD14 or sCD14 deletion mutant is shown on the Y-axis; time is shown on the X-axis. Two separate experiments are superimposed. Wild-type sCD14 is depicted by circles; FITC-LPS is added at 0 seconds and the protein is added at 70 seconds. DDED/PQPD is shown as a line without symbols; FITC-LPS is added at 50 seconds and the protein is added at 120 seconds. The abrupt declines in the baseline are due to the opening of the shutter to add reagents.

FIG. 4 shows a sucrose density gradient analysis of ³H-LPS:CD14 complexes is shown: cpm (³H-LPS) are shown on the Y-axis; fractions are shown on the X-axis. Fraction 1 corresponds to the bottom of the tube. The PQPD, AVEVE and DDED/PQPD mutants were indistinguishable from wild-type CD14. This represents one of two experiments with identical results.

FIG. 5 shows the IL-6 response of U373 cells to 10 ng/ml Re595 LPS as a function of increasing concentrations of sCD14 deletion mutant. Each point is the average of two determinations; the range is smaller than the symbols. Open circles, wild-type CD14; closed circles, DDED deletion; open triangles, PQPD deletion; closed triangles, DDED/PQPD deletion; open squares, DPRQY deletion; closed squares, AVEVE deletion. This is one of three experiments with similar results.

FIG. 6, panel A shows the IL-6 response of U373 cells to 10 ng Re595 LPS in the presence of 10 mg (open bars) or 50 mg (closed bars) of the indicated deletion mutants is shown. The bars represent the average of two determinations and the error bars represent the range.

IL-6 was not produced in response to CD14 or mutant CD14 unless LPS was added.

FIG. 6, panel B shows the IL-6 response of U373 to 10 ng Re LPS and 200 ng/ml sCD14 in the presence of increasing concentrations of sCD14 mutant. The symbols represent the average of two determinations and the error bars represent the range. Open circles, DDED; closed circles, PQPD; open triangles, DDED/PQPD; closed triangles, DPRQY; open squares, AVEVE. The line represents the IL-6 response to 10 ng/ml LPS and 200 ng/ml sCD14 in the absence of any mutant CD14. This is one of five experiments with similar results.

FIG. 7 shows the IL-6 response of U373 cells to 10 ng/ml Re LPS in the presence of 0.5% serum. The symbols represent the average of two determinations and the error bars represent the range. Open circles, PQPD; closed circles, DDED/PQPD; open triangles, AVEVE. The line represents the response to 10 ng/ml RE LPS and 0.5% serum in the absence of any mutant CD14. This is one of two similar experiments.

FIG. 8 shows amino acid structural conservation of various mammalian CD14 molecules using a Clustal W sequence alignment program (see http://www.genome.ad.jp/SIT/CLUSTALW.html) using the following NCBI GenBank accession sequences and corresponding nucleic acid counterparts: human: AAA51930 (nucleic acid accession #M86511); mouse: CAA32166 (nucleic acid accession #X13987), rabbit: BAA21770 (nucleic acid accession #D16545); and bovine: BAA21517 (nucleic acid accession #D84509). Those of ordinary skill in the art can identify and manipulate other mammalian species using these and other common resources and techniques.

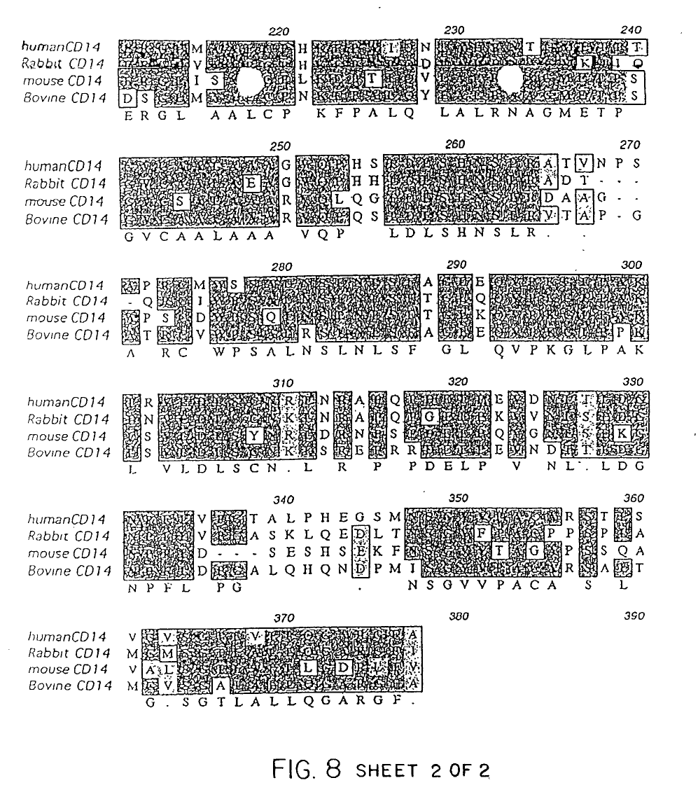

### DETAILED DESCRIPTION

Definitions

By “domain” or “region” is meant a cluster of amino acid residues in a linear or folded polypeptide that correlates with a given function. The component residues need not be contiguous to one another, e.g., in the instance of a properly folded, biologically active polypeptide or protein molecule. In such instance, amino acids well separated in a linear context (primary array) are brought into close juxtaposition by virtue of folding and conformation associated with biological activity of the native molecule (secondary and tertiary arrays). Domains may be relatively near or distant from one another and capable of description in terms of primary, secondary, and tertiary arrays, or any combination thereof.

Binding at one domain can exert functional influence at another domain, e.g., in the case of human CD14, as the invention attests. However, multiple domains within a polypeptide or protein may also be capable of being decoupled, both physically and functionally from one another, e.g., by excising surrounding regions and domains. In decoupled form, the domains may still exert, more or less, the same intrinsic function they possessed when present in the larger multi-domain molecule.

As used herein, a “binding domain” or the “first domain” of the present invention includes but is not limited to amino acid residues 58-62, and subsets therein, of the human CD14 molecule and corresponding residues in related mammalian structures.

A “domain associated with transmission of a cellular signal”, e.g., in the case of the CD14 receptor, refers to residues from about 1-54 and subsets thereof, particularly residues 8-11 and 21-24 of the human CD14 receptor (and mammalian equivalents thereof) that the applicants have found to collectively exert an effect on binding at the downstream binding (i.e., “first”) domain. This definition does not preclude the later identification and inclusion of other contributing residues that may be present in neighboring protein folds not exactly within residues 1-54.

By “enhanced affinity” is meant an increased propensity to bind, e.g., an LPS molecule to a CD14 receptor or domain thereof. For such a relation, a steady state is achieved in the presence of fixed amounts of ligand (e.g., LPS molecule) and receptor (e.g., CD14 receptor) such that bound and unbound species exist at any given time and are measurable. An enhanced affinity denotes a tighter or more frequent binding of an equimolar amount of modified receptor relative to unmodified receptor in the presence of a fixed amount of ligand at any given time. Those of skill in the art are familiar with kinetic parlance and experimentation to describe and quantify binding affinities.

By “foreign body” is meant a microbe including but not limited to a gram negative or gram-positive bacterium, or fungi, that can invade and infect a larger host body or organism. By “membrane component from a foreign body” is meant a molecular entity residing in the microbe's membrane or cell wall, preferably lipid in nature or content, and capable of binding both native CD14 receptors and modified CD14 receptors of the present invention in vivo, and in simulated environments ex vivo, e.g., in tissue culture experiments employing cell and biochemical techniques commonly known to those skilled in the art. Examples of such components include but are not limited to lipopolysaccharides (LPS) and peptidoglycans, characteristic respectively of gram-negative and gram-positive bacteria.

By “gram-negative” bacteria is meant those bacteria having a plurality of exterior membranes, a distinctive outer membrane component of which is lipopolysaccharide (LPS) capable of binding to a mammalian host's native CD14 receptors and thereby inducing disease etiology and symptoms characteristic of microbe infection. Typical gram-negative species contemplated for the invention include but are not limited to those most commonly associated with sepsis and septic shock in humans, e.g., as reported in the HANDBOOK OF ENDOTOXINS, 1: 187-214, eds. R. Proctor and E. Rietschel, Elsevier, Amsterdam (1984).

By “gram-positive” bacteria is meant bacteria characterized by a preponderance of peptidoglycans relative to LPS molecules in their membranes, which are capable of binding to a mammalian host's native CD14 receptors and thereby inducing disease etiology and symptoms characteristic of microbe infection, similar to those described for gram-negative species.

The terms “modified” and “altered” and grammatical permutations thereof are used synonymously to denote change. The change may be a structural change, e.g., as in the deletion of various amino acid residues within a larger peptide or protein sequence, and/or a functional change, e.g., as in the variation of cellular signal transmission or ligand (LPS) binding.

Secondary and tertiary “structures” contribute greatly to biological function. However, the term “structural” as used herein may also denote a lesser and more easily understood concept, such as a primary linear array of amino acid residues, e.g., in a denatured peptide or protein.

By “remote” is meant separated from, as in the individual and relative position of amino acid residues in a polypeptide or protein array, whether such array is primary, secondary, or tertiary as those terms are known and understood by those skilled in the art. The concept is thus relative and does not preclude some measure of proximity, as in the cooperative interaction of multiple distinct residues or domains within a properly folded, biologically active polypeptide or protein molecule which constituent residues would otherwise be much further apart if considered in a linear, primary sense. In other words, what in linear terms might amount to a substantial distance between domains or certain individual residues within a polypeptide or protein can assume a much closer relative position in vivo (i.e., when a polypeptide or protein adopts “secondary” and “tertiary” structures as commonly understood in the art). Of course, and as is also well understood in the art, in vivo conditions may be sufficiently simulated in vitro or ex vivo so as to afford logical inference concerning in vivo structure and function. The term “remote” is thus a flexible one.

By “essentially unmodified” or “essentially unchanged” means that the particular domain, receptor, or function thereof with which the term is associated, would otherwise be structurally intact or functionally competent but for the existence of an introduced modification thereto. This can mean that the receptor is substantially full-length, as in its appearance relative to a naturally existing receptor analog in nature.

As used herein, “substantially full-length” denotes a CD14 receptor having at least the first N-terminal 153 amino acid residues of the native receptor, with additional allowance for minor deletions such as those noted herein that contribute to enhanced binding ability of the modified receptor.

By “pharmaceutical composition” is meant one that is suitable for administration to an animal, preferably a mammal, more preferably a rat or mouse, and most preferably a primate such as a human. The composition is preferably sterile and includes one or more components designed to effect a particular result in a particular organism when administered. The administration means is variable and intended to especially suit the particular affliction, taking into account such factors as host health and other characteristics. The component or components within the composition, and as concerns the modified CD14 molecules of the present invention, preferably include(s) a purified version of the CD14 molecule, or a nucleic acid sequence encoding such and capable of expression in the host to which administration is to be made. Thus, the term “included as part of a pharmaceutical composition”, besides referring to the event in which actual CD14 is supplied a protein or polypeptide, also encompasses the situation wherein the actual protein is produced in vivo upon supply and delivery of an underlying nucleic acid sequence encoding such.

By “treatment” is meant an amelioration or alleviation of symptoms including, but not limited to, complete elimination of those symptoms from a host organism. Thus, the term also embraces lesser degrees short of complete cure, including positive therapeutic effects of any degree.

By “symptom or symptoms associated with sepsis or septic shock” includes, but is not limited to, one or more of the following: hypotension, chills, profuse sweating, fever, weakness, leukopenia, intravascular coagulation, shock, respiratory distress, organ distress or failure, and prostration.

The discussion that follows addresses general aspects and embodiments of the invention that are largely prophetic but readily enabling, alone, or in combination with what is known in the art. From there, specific experimental examples that have been performed are provided.

Generation and Administration of Pharmaceutical Compositions

Those of skill in the art are familiar with the principles and procedures discussed in such widely known and available sources as Remington's Pharmaceutical Science, 17th Ed., Mack Publishing Co., Easton Pa. (1985) and Goodman and Gilman 's The Pharmacological Basis of Therapeutics, 8th Ed., Pergamon Press, Elmsford, N.Y. (1990), each of which is herein incorporated by reference.

Generally, compositions of the invention will comprise a therapeutically effective amount of modified mammalian CD14 receptor in a pharmaceutically acceptable carrier or excipient. Such a carrier includes but is not limited to saline, buffered saline, dextrose, water, glycerol, ethanol, and combinations thereof. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Various delivery systems are known and can be used to administer a therapeutic of the invention, e.g., encapsulation in liposomes, microparticles, microcapsules and the like.

The composition is formulated in accordance with routine procedures as a pharmaceutical composition adapted for intravenous administration to human beings or other mammals. Typically, compositions for intravenous administration are solutions in sterile isotonic aqueous buffer. Where necessary, the composition may also include a solubilizing agent and a local anesthetic to ameliorate any pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The therapeutics of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-4000 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

A cogent useful extrapolation can be made to results published in U.S. Pat. No. 5,804,189, wherein human recombinant soluble (unmodified) CD14 was effectively administered intravenously to mice at a concentration of about 68 ug/20 g or ^{˜}3.4 mg/kg body weight. It will generally be desirable to back-titrate to determine the least amount of administered receptor that results in a positive effect without unacceptable side-effects. Similar concentrations can then be used in human clinical trials.

Those of skill in the art, applying routine pharmacological techniques, can readily determine a suitable formulation without exercising undue experimentation.

Genetically Based Alternatives to Direct Protein Administration

Because sepsis, if not treated accurately and promptly, can rapidly be fatal to its host victim, it must be treated quickly. Thus, administration of the pre-fabricated polypeptide is preferred over other means. However, the pharmaceutical compositions of the invention need not be supplied in peptide or protein form, but instead may be administered as a nucleic acid species which can then be conveniently expressed, preferably quickly and transiently, in the afflicted host. This is especially so in versions of the modified receptor that do not contemplate extraneous chemical modification that is not programmable at the nucleotide level. For example, those of skill know that certain destabilizing amino acid sequences can be introduced into a peptide, e.g., targeted protease cleavage points, such that the overall peptide is more readily degraded and does not persist to generate unwanted side-effects.

In satisfaction of the preferred embodiment wherein modified CD14 receptor is present in a patient's body no longer than necessary to fulfill its intended function, and is then conveniently eliminated, it will be recognized that genetic contructs capable of perpetual self-replication, chromosomal integration, or replication otherwise coordinated with a cell's own replication are not desired unless expression can be tightly controlled or regulated so as to completely shut production of the protein on demand, such as by chemical or temperature inducement. Therefore, in convenient applications related to the invention, it is envisioned that the genetic construct be capable of transient expression only, and that to the degree such expression is inadequate to completely fulfill the desired therapeutic function, additional transiently expressing constructs be administered to supplement or conclude the action.

The genetic constructs contemplated will embody any combination of DNA, RNA, hybrids thereof (referred hereinafter as nucleic acids) or chemically modified derivatives thereof that are operably linked to regulatory elements, e.g., promoters, enhancers, polyadenylation sequences, Kozak sequences, including initiation and stop codons, etc., needed for gene expression.

Accordingly, incorporation of the DNA or RNA molecule into a living cell results in the expression of the DNA or RNA encoding modified CD14, and production of the protein. Because the activity of CD14 is extracellular, a secretory signal must be employed to shunt active CD14 to the cell exterior. Such a signal is normally encoded as a series of N-terminal amino acids. The secretory signal used may be the innate CD14 receptor gene's own signal, or other more or less effective signals.

Examples of promoters that can be used to produce to the modified mammalian CD14 receptors of the instant invention, e.g., in humans, include but are not limited to promoters from Simian Virus 40 (SV40), Mouse, Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from other human genes such as human Actin, human Myosin, human Hemoglobin, human muscle creatine and human metalothionein. Obviously, innate human CD14 promoter sequences may also be used.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal which is in pCEP4 plasmid (Invitrogen, San Diego Calif.), referred to as the SV40 polyadenylation signal, can be used.

Examples of alternative enhancers may be selected from the group including but not limited to: human Actin, human Myosin, human Hemoglobin, human muscle creatine and viral enhancers such as those from CMV, RSV and EBV.

In some preferred therapeutic embodiments, an additional element may be added which serves as a target for cell destruction if it is desirable to eliminate cells receiving the genetic construct for any reason, e.g., to only transiently produce the modified CD14. For example, a herpes thymidine kinase (tk) gene in an expressible form can be included as part of the same genetic operon or construct. After the modified CD14 has fulfilled its function, the drug gangcyclovir can then be administered to the individual and that drug will cause the selective killing of any cell producing tk, thus, providing the means for the selective destruction of cells with the genetic construct.

In order to maximize protein production, regulatory sequences may be selected which are well suited for gene expression in the cells the construct is administered into. Moreover, codons may be selected which are most efficiently transcribed in the cell or tissue type, or mammalian host of interest, generally.

The genetic therapeutic may be administered directly into the individual or ex vivo into removed cells of the individual which are reimplanted after administration. By either route, the genetic material is introduced into cells which are present in the body of the individual Routes of administration include, but are not limited to, intramuscular, intraperitoneal, intradermal, subcutaneous, intravenous, intraarterial, intraoccular, and oral, as well as transdermally or by inhalation or suppository. Preferred routes of administration include intramuscular, intraperitoneal, intradermal and subcutaneous injection. Delivery of gene constructs which encode target proteins can confer mucosal immunity in individuals immunized by a mode of administration in which the material is presented in tissues associated with mucosal immunity. Thus, in some examples, the gene construct is delivered by administration in the buccal cavity within the mouth of an individual. Genetic constructs may be administered by means including, but not limited to, traditional syringes, needleless injection devices, or “microprojectile bombardment gene guns”. Alternatively, the genetic vaccine may be introduced by various means into cells that are removed from the individual. Such means include, for example, ex vivo transfection, electroporation, microinjection and microprojectile bombardment. After the genetic construct is taken up by the cells, they are reimplanted into the individual. It is contemplated that otherwise non-immunogenic cells that have genetic constructs incorporated therein can be implanted into the individual even if the vaccinated cells were originally taken from another individual.

The genetic vaccines according to the present invention comprise about 1 nanogram to about 1000 micrograms of DNA. In some preferred embodiments, the vaccines contain about 10 nanograms to about 800 micrograms of DNA. In some preferred embodiments, the vaccines contain about 0.1 to about 500 micrograms of DNA. In some preferred embodiments, the vaccines contain about 1 to about 350 micrograms of DNA. In some preferred embodiments, the vaccines contain about 25 to about 250 micrograms of DNA. In some preferred embodiments, the vaccines contain about 100 micrograms DNA. The genetic vaccines according to the present invention are formulated according to the mode of administration to be used. One having ordinary skill in the art can readily formulate a genetic vaccine or therapeutic that comprises a genetic construct.

In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present invention are preferably provided sterile and pyrogen free.

Generation of Chimeric CD14/Fc Molecules

Further embodiments contemplate chimeric proteins comprised of CD14 fragments and COOH-attached Fc portions from immunoglobulins, e.g., IgM or IgG. This theoretically will improve the valence and avidity of microbe membrane components such as LPS for the novel modified receptors described herein, thereby further enhancing their utility as pharmaceutical blocking agents and diagnostic implements. Illustrative applications are found in U.S. Pat. No. 5,981,724, herein incorporated by reference, and in Fanslow et al., J. Immunol. 149:65, (1992) and Noelle et al., Proc. Natl. Acad. Sci. U.S.A. 89:6550, (1992). As applied, because it is known that the first 150 or so amino acids of the CD14 receptor are required for overall activity, preferred embodiments will at least retain this portion, and join the Fc immunoglobulin portion in-frame to the COOH terminal end of the functional CD14 molecule. Ideally, this joinder should not functionally interfere with the CD14 portion of the molecule in vivo or in vitro. Such constructions can be made directly at the peptide level, using synthetic peptide linkages and chemistry, or more preferably at the nucleotide level by simply joining the underlying sequences in-frame, and amplifying them, e.g., using PCR and primers specific for each end of the hybrid nucleic acid molecule. The ampified chimeric species can then be placed conveniently into an expression vector or cassette as known in the art and delivered as a therapeutic, or else expressed in vitro using, e.g., using the baculovirus system described herein, and the chimeric product isolated therefrom. Alternative “tags” for purification and diagnostic purposes Described in detail is a peptide bearing a 6His carboxy “tag” entity to facilitate protein purification (affinity chromatography). Alternative or additional tags can also be employed and are embraced within the scope of this invention.

For example, the Flag™ octapeptide (Hopp et al., Bio/Technology 6:1204, 1988; offered through Kodak, New Haven, Conn.) can be positioned at the N-terminus and does not alter the biological activity of fusion proteins, is highly antigenic and provides an epitope reversibly bound by a specific monoclonal antibody, enabling rapid detection and purification of the expressed fusion protein. The sequence is also specifically cleaved away by bovine mucosal enterokinase. A murine monoclonal antibody that binds the Flag sequence has been deposited with the ATCC under accession number HB 9259. Methods of using the antibody in purification of fusion proteins comprising the Flag sequence are described in U.S. Pat. No. 5,011,912, which is incorporated by reference herein.

Other types of linkers that can be used include, but are not limited to maltose binding protein (MBP), glutathione-S-transferase (GST), thioredoxin (TRX) and calmodulin binding protein (CBP). Kits for expression and purification of such fusion proteins are commercially available from, e.g., New England BioLabs (Beverly, Mass.), Pharmacia

(Piscataway, N.J.) InVitrogen (Carlsbad, Calif.) and Stratagene (San Diego, Calif., respectively.

In addition, it may be necessary to add between the individual portions of the hybrid protein a “linker” or “spacer” as is known in the art to ensure that the proteins form proper secondary and tertiary structures so as to endow the full-length molecule to be functional as a CD14 receptor. Suitable linker sequences will adopt a flexible extended conformation, will not exhibit a propensity for developing an ordered secondary structure which could interact with the functional domains of fusion proteins, and will have minimal hydrophobic or charged character which could promote interaction with the functional protein domains. Typical surface amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, may also be used in the linker sequence. The length of the linker sequence may vary without significantly affecting the biological activity of the fusion protein. Exemplary linker sequences are described in U.S. Pat. Nos. 5,073,627 and 5,108,910, herein incorporated by reference.

Conventional Purification

Described above are affinity chromatography methods of purification. Not to be overlooked as alternative or combined methodologies are those employing conventional and different means of purification.

For example, supernatants from systems which secrete recombinant protein into culture media may be first concentrated using a commercially available protein concentration filter, such as an Amicon or Millipore Pellicon ultrafiltration unit. Following the concentration step, the concentrate may be applied to a suitable purification matrix. For example, a suitable affinity matrix may comprise a counter structure protein (i.e., a protein to which a polypeptide binds in a specific interaction based on structure) or antibody molecule bound to a suitable support. Alternatively, or conjunctively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Sulfopropyl groups are preferred. Gel filtration chromatography also provides a means of purifying polypeptides.

Alternative or Additional Modifications to the Receptor

The Applicants have identified particular regions of importance in the CD14 receptor that when jointly manipulated endow enhanced binding affinity at a remote locus on the receptor. Deletions were used to demonstrate this. However, it is envisioned that other changes, such as substitutions and insertions, will also work when applied to these general regions. Furthermore, neutral or conservative additions may be made to the surrounding residues within the CD14, e.g., conservative substitutions, so as to preserve receptor function, and especially the increased binding affinity associated with the modified receptors of the instant invention. A “conservative substitution” in the context of the subject invention is one in which an amino acid is substituted for another amino acid that has similar properties, such that one skilled in the art of peptide chemistry would expect the secondary structure and hydropathic nature of the polypeptide to be substantially unchanged for these regions. Other such conservative substitutions, for example, include substitutions of entire regions having similar hydrophobicity characteristics, are well known. Mutagenic techniques for such replacement, insertion or deletion are well known to those skilled in the art (see, e.g., U.S. Pat. No. 4,518,584).

Chemical Synthesis of CD14 Backbone Polypeptide

Although not preferred, polypeptides of this invention may also be prepared synthetically. Synthetic formation of the polypeptide or protein requires chemically synthesizing the desired chain of amino acids by methods well known in the art. Chemical synthesis of a peptide is conventional in the art and can be accomplished, for example, by the Merrifield solid phase synthesis technique [Merrifield, J., Am. Chem. Soc., 85: 2149-2154 (1963); Kent et al., Synthetic Peptides in Biology and Medicine, 29 f.f. eds. Alitalo et al., (Elsevier Science Publishers 1985); and Haug, J. D., “Peptide Synthesis and Protecting Group Strategy”, American Biotechnology Laboratory, 5 (1): 40-47 (January/February. 1987)]. Techniques of chemical peptide synthesis include using automatic peptide synthesizers employing commercially available protected amino acids, for example, Biosearch [San Rafael, Calif. (USA)] Models 9500 and 9600; Applied Biosystems, Inc. [Foster City, Calif. (USA)] Model 430; Milligen [a division of Millipore Corp.; Bedford, Mass. (USA)] Model 9050; and Du Pont's RAMP (Rapid Automated MultiplePeptide Synthesis) [Du Pont Compass, Wilmington, Del. (USA)]. Generally, however, such synthesis is expensive, and with limitations in the length of the peptides which can be produced (˜50-100 amino acid residues), and therefore is not preferred Allowance is made, however, for advances in the field that might facilitate or promote this means of synthesis in use of the invention. General guidance may be found in Merrifield, J. Am. Chem. Soc. 85-2149-2146, 1963).

Derivatization

Whether synthesis is performed chemically or recombinantly, it may be desirable to further modify the polypeptide backbone prior to use as a diagnostic or therapeutic agent.

Covalent modifications of the protein or peptide are included within the scope of this invention. Such modifications may be introduced into the molecule by reacting targeted amino acid residues of the peptide with an organic derivatizing agent that is capable of reacting with selected side chains or terminal residues.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, .alpha.-bromo-.beta.(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Parabromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect or reversing the charge of the lysinyl residues. Other suitable reagents for derivatizing .alpha.-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high PK of the guanidine functional group. Furthermore, these reagents may react with the groups of lysine as well as the arginine .epsilon.-amino group.

Tyrosyl residues are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction carbodiimide (R′—N—C—N—R′) such as 1-cyclohexyl-3-(2-morpholinyl(4-ethyl) carbodiimide or 1-ethyl-3-(4-azonia-4,4-dimethylpentyl) carbodiimide. Furthermore, aspartyl and glutamyl residue are converted to asparaginyl and glutaminyl residues by reaction with ammonium ions. Glutaminyl and asparaginyl residues are frequently deamidated to the corresponding glutamyl and aspartyl residues. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the alpha-amino groups of lysine, arginine, and histidine side chains (Creighton, T. E., PROTEINS: STRUCTURE AND MOLECULAR PROPERTIES, W. H. Freeman & Co., San Francisco, pp. 79-86 (1983)), acetylation of the N-terminal amine, and, in some instances, amidation of the C-terminal carboxyl groups.

Such derivatized moieties may improve the solubility, absorption, biological half life, and the like. The moieties may alternatively eliminate or attenuate any undesirable side effect of the protein. For additional useful discussion, the reader is directed to Remington's Pharmaceutical Sciences, 16th ed., Mack Publishing Co., Easton, Pa. (1980), herein incorporated by reference.

Attachment to Solid-Support Surfaces

Derivatization with bifunctional agents is useful for cross-linking polypeptides to a water-insoluble support matrix or to other macromolecular carriers in preparation for affinity chromatography and other diagnostic and/or purification procedures. Commonly used cross-linking agents include, for example, 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl) dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

The linkages need not be mediated by covalent bonding, but may alternatively or conjunctively employ strong noncovalent bonding means (e g., streptavidin-biotin) known to those of skill in the art.

Antibody Production Against Modified CD14 Receptors

As will be demonstrated below in the specific examples, native CD14 has a given “fingerprint” or profile in terms of known antigenic determinants or epitopes recognized by known antibodies. Modification of the CD14 receptor can vary this pattern, as the invention demonstrates. Specifically, the introduced modifications may eliminate certain epitopes, and may introduce certain others anew. These new epitopes may be useful for generating novel and specific antibodies that may be further useful in purification, therapeutic, and diagnostic procedures employing the modified receptors. It is therefore appropriate to elaborate on how the new antibodies may be produced. As will be appreciated by one of ordinary skill in the art, antibodies may be developed which not only bind, but which also block biological activity associated with that, or a neighboring, epitopic determinant.

Polyclonal antibodies may be readily generated by one of ordinary skill in the art from a variety of warm-blooded animals such as horses, cows, various fowl, rabbits, mice, or rats. Briefly, the modified CD14 receptor is utilized to immunize the animal through intraperitoneal, intramuscular, intraocular, or subcutaneous injections, an adjuvant such as Freund's complete or incomplete adjuvant. Following several booster immunizations, samples of serum are collected and tested for reactivity modified CD14. Particularly preferred polyclonal antisera will give a signal on one of these assays that is at least three times greater than background. Once the titer of the animal has reached a plateau in terms of its reactivity, larger quantities of antisera may be readily obtained either by weekly bleedings, or by exsanguinating the animal.

Monoclonal antibodies may also be readily generated using conventional techniques (see U.S. Pat. Nos. RE 32,011, 4,902,614, 4,543,439, and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference).

Briefly, within one embodiment a subject animal such as a rat or mouse is injected with modified CD14. The CD14 may be admixed with an adjuvant such as Freund's complete or incomplete adjuvant in order to increase the resultant immune response. Between one and three weeks after the initial immunization the animal may be reimmunized with another booster immunization, and tested for reactivity using assays described above. Once maximum reactivity is achieved, the animal is sacrificed, and organs which contain large numbers of B cells such as the spleen and lymph nodes are harvested. Cells which are obtained from the immunized animal may be immortalized by transfection with a virus such as the Epstein bar virus (EBV) (see Glasky and Reading, Hybridoma 8(4):377-389, 1989). Alternatively, within a preferred embodiment, the harvested spleen and/or lymph node cell suspensions are fused with a suitable myeloma cell in order to create a “hybridoma” which secretes monoclonal antibody.

Suitable myeloma lines include, for example, NS-1 (ATCC No. TIB 18), and P3X63-Ag 8.653 (ATCC No. CRL 1580). Following the fusion, the cells may be placed into culture plates containing a suitable medium, such as RPMI 1640, or DMEM (Dulbecco's Modified Eagles Medium) (JRH Biosciences, Lenexa, Kans.), as well as additional ingredients, such as Fetal Bovine Serum (FBS, ie., from Hyclone, Logan, Utah, or JRH Biosciences). Additionally, the medium should contain a reagent which selectively allows for the growth of fused spleen and myeloma cells such as HAT (hypoxanthine, aminopterin, and thymidine) (Sigma Chemical Co., St. Louis, Mo.). After about seven days, the resulting fused cells or hybridomas may be screened in order to determine the presence of antibodies which are reactive against CD14.

A wide variety of assays may be utilized to determine the presence of antibodies which are reactive against modified CD14, including for example Countercurrent Immuno-Electrophoresis, Radioimmunoassays, Radioimmunoprecipitations, Enzyme-Linked Immuno-Sorbent Assays (ELISA), Dot Blot assays, Inhibition or Competition Assays, and sandwich assays (see U.S. Pat. Nos. 4,376,110 and 4,186,530; see also Antibodies: A Laboratory Manual, Harlow and Lane (eds.), Cold Spring Harbor Laboratory Press, 1988). Following several clonal dilutions and re-assays, a hybridoma producing antibodies reactive against modified CD14 may be isolated. By comparing reactivity to other known reactive antibodies to native CD14, such as those described herein, one may identify antibodies specific to the “modified” receptor.

Other techniques may also be utilized to construct monoclonal antibodies (see, e.g., Huse et al., “Generation of a Large Combinational Library of the Immunoglobulin Repertoire in Phage Lambda,” Science 246:1275-1281, December 1989; see also L. Sastry et al., “Cloning of the Immunological Repertoire in Escherichia coli for Generation of Monoclonal Catalytic Antibodies: Construction of a Heavy Chain Variable Region-Specific cDNA Library,” Proc Natl. Acad. Sci USA 86:5728-5732, August 1989; see also Michelle Alting-Mees et al., “Monoclonal Antibody Expression Libraries: A Rapid Alternative to Hybridomas,” Strategies in Molecular Biology 3:1-9, January 1990.

Labeling of Modified CD14 and Specific Antibodies Thereof

Modified CD14 receptor, derivatives thereof, and antibodies thereto may be labeled with a variety of molecules, including, e.g., fluorescent molecules, substances having therapeutic activity i e therapeutic agents, luminescent molecules, enzymes, and radionuclides. Representative examples of fluorescent molecules include fluorescien, phycoerythrin, rodamine, Texas red and luciferase. Representative examples of radionuclides include Cu-64, Ga-67, Ga-68, Zr-89, Ru-97, Tc-99m, Rh-105, Pd-109, In-111, I-123, I-125, I-131, Re-186, Re-188, Au-198, Au-199, Pb-203, At-211, Pb-212 and Bi-212. Examples of suitable enzymes include horseradish peroxidase, biotin, alkaline phosphatase, beta.-galactosidase, or acetylcholinesterase; and an example of a luminescent luminol. In addition, the modified CD14 or antibodies specific thereto may also be labelled or conjugated to one partner of a ligand binding pair. Representative examples include avidin-biotin, and riboflavin-riboflavin binding protein. Methods for conjugating or labeling the modified CD14 or specific antibodies thereto are known to those of skill and procedures directed thereto further elaborated in U.S. Pat. Nos. 4,744,981, 5,106,951, 4,018,884, 4,897,255, and 4,988,496; see also Inman, Methods In Enzymology, Vol. 34, Affinity Techniques, Enzyme Purification: Part B, Jakoby and Wichek (eds.), Academic Press, New York, p. 30, 1974; see also Wilchek and Bayer, “The Avidin-Biotin Complex in Bioanalytical Applications,” Anal. Biochem. 171 :1-32, 1988).

Diagnostic Kits and Assays

According to another aspect of the present invention, there is provided a diagnostic kit for detecting the presence of microbial particles, e.g., LPS and/or peptidoglycans, in samples including serum taken from normal donors, patients suspected of having disease, and patients with diseases who are being monitored throughout the course of treatment or remission. Levels of microbe particles detected by the binding assay that are significantly higher than the baseline levels of a statistically significant population size of known normal donors without evidence of active disease are considered to be indicative of active disease. The kit, according to the present invention, can include a variety of compounds and reagents in addition to a supply of modified CD14 receptor, preferably pre-bound to a solid-support, or with instructions for how to do so.

Formats derive from classic antibody:antigen/receptor:ligand assays as described in Harlow and Lane, Antibodies: A Laboratory Manual, Cold Spring Harbor Laboratory, 1988, herein incorporated by reference. In preferred embodiments, CD14 is immobilized on a solid support and biological sample introduced which may have suitable microbe ligand. Bound ligand is then detected using a detection reagent that binds to the complex and contains a reporter group. Suitable detection reagents include labeled antibodies or free, labeled CD14 The extent which the components of the sample inhibit the binding of the labeled entity is indicative of the reactivity of the sample with the immobilized CD14.

The solid support may be any material known to those of ordinary skill in the art to which the polypeptide CD14 may be attached. For example, the support may be a test well in a microtiter plate or nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, e.g., in U.S. Pat. No 5,359,681.

Other means include, e.g., nickel-plated or associated “chips” capable of binding a 6his tag of the modified receptor, which then in turn is capable of binding ligand (eg, LPS), and the latter binding capable of evaluation using hardware and methodologies that exist in the market place and are known to those of skill in the art (e.g., BIACORE or SELDI procedures and instrumentation) At least in the case of SELDI, ligand MW can be determined via mass spectrometry and this information relative to known standards used to “type” or “fingerprint” different ligands, thereby indicating the identity of the microbe or microbe type responsible for infection.

Binding of the polypeptide receptor is not limited to the above, and may be accomplished using any of various techniques known to those in the art. These techniques are amply described in the patent and scientific literature. The binding may be by noncovalent association, such as adsorption, or covalent attachment. Covalent attachment may be accomplished by a direct linkage between the antigen and functional groups on the support or by way of a cross-linking agent, as discussed above. Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with a suitable amount of polypeptide. Covalent attachment of polypeptide to a solid support may generally be achieved by first reacting the support with a bifunctional reagent that will react with both the support and a functional group, such as a hydroxyl or amino group, on the polypeptide. For example, the polypeptide may be bound to a support having an appropriate polymer coating using benzoquinone or by condensation of an aldehyde group on the support with an amine and an active hydrogen on the polypeptide (see, e.g., Pierce Immunotechnology Catalog and Handbook (1991) at A12-A13); see also section below on conjugation to solid-supports.

In some preferred embodiments, the assay is an enzyme linked immunosorbent assay (ELISA) as known in the art. This assay may be performed by first contacting a polypeptide (e.g., CD14 receptor) that has been immobilized on a solid support, commonly the well of a microtiter plate, with the sample such that microbe particles within the sample are allowed to bind to the immobilized polypeptide. Sample containing unbound microbe particles is then removed from the immobilized polypeptide, and a detection reagent capable of binding to the immobilized complex is added. The amount of detection reagent that remains bound to the solid support is then determined using a method appropriate for the specific detection reagent. Once the polypeptide is immobilized on the support, the remaining protein binding sites on the support are typically blocked. Any suitable blocking agent known to those of ordinary skill in the art, such as bovine serum albumin (BSA), non-fat dry milk, or Tween 20.TM. (Sigma Chemical Co., St. Louis, Mo.) may be employed. The immobilized polypeptide is then incubated with the sample, and microbe particles (if present in the sample) allowed to bind. The sample may be diluted with a suitable diluent, such as phosphate-buffered saline (PBS) prior to incubation. In general, an appropriate contact time (i.e., incubation time) is that period of time that is sufficient to detect the presence of microbe.

The unbound sample is then washed away (e.g. in PBS containing 0.1% Tween 20) and detection reagent added. An appropriate detection reagent is any compound that binds to the immobilized complex and that can be detected by any of a variety of means known to those in the art. Preferably, the detection reagent is bound to a reporter, e.g, an enzyme such as horseradish peroxidase, a dye, radioisotope, luminescent molecule, fluorescent molecule or biotin. The conjugation of binding agent to reporter group may be achieved using standard methods known to those of ordinary skill in the art. Common binding agents may also be purchased conjugated to a variety of reporter groups (e.g., Zymed Laboratories, San Francisco, Calif. and Pierce, Rockford, Ill.). The detection reagent is then incubated with the immobilized complex for an amount of time sufficient to detect the bound ligand. An appropriate amount of time may generally be determined from the manufacturer's instructions or by assaying the level of binding that occurs over a period of time. Unbound detection reagent is then removed, and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent molecules and fluorescent molecules. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence and amount of microbe particles in a sample, the signal detected from the reporter group that remains bound to the solid support is generally compared to a signal that corresponds to a predetermined cut-off value. In one preferred embodiment, the cut-off value is preferably the average mean signal obtained when the immobilized polypeptide is incubated with samples from an uninfected patient. In general, a sample generating a signal that is three standard deviations above the predetermined cut-off value is considered positive (i.e., reactive with the polypeptide).

In related embodiments, the assay is performed in a flow-through or strip test format, wherein modified CD14 is immobilized on a membrane such as nitrocellulose. In the flow-through test, microbe particles, e.g., LPS within the sample, bind to the immobilized polypeptide as the sample passes through the membrane. A detection reagent then binds to the complex as the solution containing the detection reagent flows through the membrane. The detection of bound detection reagent may then be performed as described above.

In a strip test format, one end of the membrane to which polypeptide is bound is immersed in a solution containing the sample. The sample migrates along the membrane through a region containing detection reagent and to the area of immobilized polypeptide. Concentration of detection reagent at the polypeptide indicates the presence of microbe in the sample. Typically, the concentration of detection reagent at that site generates a pattern, such as a line, that can be read visually. The absence of such a pattern indicates a negative result. In general, the amount of polypeptide immobilized on the membrane is selected to generate a visually discernible pattern when the biological sample contains a level of antibodies that would be sufficient to generate a positive signal in an ELISA, as discussed above. Such tests can typically be performed with a very small amount (e.g., one drop) of patient serum or blood.

This concludes the general discussion. Discussion will now be had of specific experimental procedures alreadey undertaken. Those of skill in the art will appreciate that numerous modifications to these procedures can be made without undue experimentation, and that numerous variations may be successfully implemented.

### EXPERIMENTAL PROCEDURES AND EXAMPLES

Reagents

Salmonella minnesota Re595 LPS (Re LPS) and fluorescent Re LPS (FITC-LPS) were prepared as previously described (26). Other types of LPS, including ³H-LPS, were obtained from List Biologicals. Monoclonal antibodies were obtained as previously described (26) or prepared from culture supernatants in our laboratory. Rabbit anti-Re LPS was prepared as described (29).

### Example 1

### Cloning and Production of Mutant CD14

CD14 cDNA was generated by Polymerase Chain Reaction (PCR) using human CD14 in pRc/RSV DNA as a template (26). The forward primer 5′-CGGATCCATGGAGCGCGCGCGTCCTGCTTGT-3′ (Seq. ID. No:2) incorporated a BamHI site and an ATG start codon into 5′ end of the DNA. The reverse primer 5′-GGGTCAGTGCTGCAACATTTTGCTGCCGGT-3′ (Seq ID. No:3) incorporated an EcoRI site into the 3′ end of the DNA fragment. The PCR reaction was performed at 68° C. annealing temperature for 30 cycles using Takara LA Taq polymerase (Pan Vera). An adapter sequence coding for six histidines (6His) with 5′ EcoRI and 3′ HindIII overhang was made by annealing the oligonucleotides, 5′-AATTCCATCACCATCACCATCACA-3′ (Seq ID. No:4) and 5′-AGCTTGTGATGGTGATGGTGATGG-3′ (Seq ID. No:5). The PCR product was cloned into the BamHI and HindIII site of the vector pFastBacI (GibcoBRL) by ligation of the PCR product digested with BamHI/EcoRI with the 6His adapter into pFastBacI DNA digested with BamHI/EcoRI. The ligation mix was transformed into E. coli DH5α.

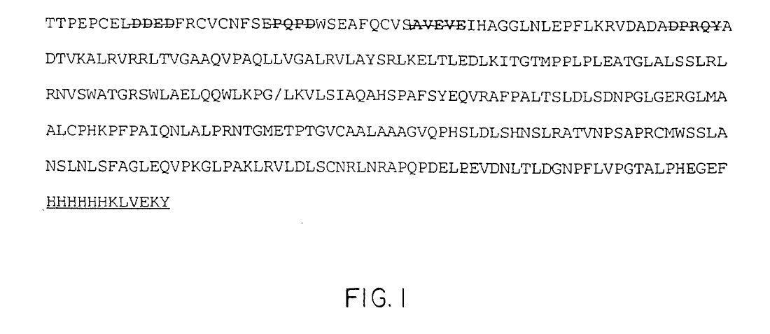

The mutant CD14 DNA was constructed by PCR using the same forward and reverse primers as the wild-type CD14 but mutant CD14 in pRc/RSV was used as the template DNA (26). The pFastBacI plasmid, containing mutant CD14 genes, was made by replacing the wild-type BamHI/EcoRI fragment from the pFastBac/CD146His with the mutant PCR BamHI/EcoRI fragment. To ensure that the constructs encoded the desired proteins, all constructs were sequenced. The resulting plasmids containing wild-type and mutant hCD14his were introduced into E. coli DH10Bac, which contained the baculovirus genome in order for homologous recombination to occur. The recombinant baculovirus DNA with the CD14 gene was then transfected into the SF9 cells (ATCC SF9 CRL 1711) to produce viral particles. The CD14 protein was produced by infecting BTI-TN-5B1-4 (High 5) cells (Invitrogen) with the virus. The High 5 cells infected with recombinant baculovirus were grown in Excel 405 serum-free medium (JRH Bioscience), and the supernatant was harvested for protein purification. The CD14 mutants described in this report are shown in FIG. 1.

The insect cell supernatant was centrifuged 10 min. at 10,000 rpm in a Sorvall RC5B centrifuge to remove debris. The supernatant was dialyzed against BV buffer (300 mM NaCl, 50 mM NaHPO₄, pH 8.0) and loaded onto a Ni-NTA column (Qiagen). The column was washed with 10 column volumes of BV buffer followed by 20 column volumes of 20 mM imidazole in BV buffer with 10% glycerol. The protein was eluted sequentially with 100 mM imidazole followed by 250 imidazole in BV buffer containing 10% glycerol. The purified protein was dialyzed against phosphate buffered saline (PBS) and concentrated with a Centriprep 30 concentrator (Amicon). The protein was analyzed for purity by SDS-PAGE and the protein concentration was determined by BCA method (Pierce).

In a few cases the 6His tag was removed by treatment of the purified protein with carboxypeptidase Y and carboxypeptidase A in PBS at a protein:enzyme ration of 1:100 for 16 h at 25° C. The 6His tag and the undigested product were removed by passing the material over a Ni-NTA column twice. The flow through was analyzed by MALDI time of flight reflectron mass spectrometry on a PerSeptive Biosystems Voyager-STR instrument. The digested products had the predicted molecular weight.

### Example 2

### Assaying Monoclonal Antibody Reactivity

CD14 and mutant CD14 were coated on a 96 well plate overnight in 0.2M acetate buffer pH 5.0 at 25° C. The wells were blocked with PBS, 1% casein for 1 hr at 37° C., and washed 3 times in PBS containing 0.05% Tween-20. The plate was incubated with various biotinylated mAbs to CD14 diluted in the blocking buffer for 1 hr at 37° C., washed 5 times in the washing buffer and incubated with streptavidin alkaline phosphatase conjugate for 1 hr at 37° C. After washing 5 times, the plate was incubated with the substrate p-nitrophenyl phosphate and the optical density (OD) determined at 405 nM. The result shown is the reactivity as compared to the wild-type.

### Example 3

### LPS Binding Assays

Three different methods were used to determine LPS-binding activity: an ELISA, a fluorescence assay, and sucrose density gradient sedimentation velocity measurements. The ELISA method was done as described with modification (30). CD14 or mutant CD14 were coated on a 96 well plate overnight in 0.2 M acetate buffer, pH 5.0. The plates were blocked with 0.5% human serum albumin (HSA), in the acetate buffer for 30 min at 37° C. Dilutions of Re LPS diluted in human serum albumin with PBS was added to each well and incubated for 10 minutes at 37° C. The plate was washed four times with PBS and affinity purified rabbit antibody to Re LPS was added (29). The plate was incubated for 1 h at 37° C. and washed with PBS. 50 ml/well of a 1:2000 dilution of anti-rabbit antibody conjugated to alkaline phosphate was added for 1 h at 37° C. The plate was washed 5 times, the alkaline phosphatase substrate added, and the OD was measured at 405 nM.

For the fluorescence assay, 10 ng/ml FITC-LPS and variable quantities of sCD14 were analyzed in a fluoremeter as published (32). The affinity was determined by using a variety of concentrations of CD14 and measuring the increase in fluorescent signal. Seven concentrations of CD14 or mutant CD14 were added to FITC-ReLPS (10 ng/ml) and the increase in fluorescence determined. The data were plotted as a double reciprocal plot (1/D fluorescence vs 1/concentration). The X-intercept was used to determine 1/K_{D}. All determinations were done twice with essentially identical results. The sucrose density gradient sedimentation velocity experiments were done by mixing 45 nM ³H LPS with 840 nM sCD14 wild-type or mutants for 15 min at 37° C. The complex was analyzed on a linear 5-20% sucrose gradient resting on a cushion of 40% sucrose as described previously (32). The gradient was centrifuged at 55,000 rpm in a VT865 rotor (Sorvall) for 80 min. 350 ml fractions were collected and the amount of ³H-LPS was determined by liquid scintillation counting. The experiments were done twice with essentially identical results.

### Example 4

### Biological Assays of sCD 14 Activity

LPS and sCD14 induction of IL-6 in U373 cells was done as described by Pugin (6). Briefly, U373 cells were cultured in a 96 well plate at a density of 5×10⁴ cells per well and grown overnight at 37° C. The cells were activated and the supernatant was harvested after 16 h. In all experiments, the CD14 was tested for its ability to stimulate cells in the absence of LPS, and it was found to be inactive. In some experiments, U373 cells were stimulated with 1 ng/ml of human IL-1 b for 16 h. The concentration of IL-6 in the supernatants was determined by ELISA. For the ELISA assay, the IL-6 was captured with goat anti-human IL-6 neutralizing antibody (R&D) and detected by rabbit anti IL-6 (Endogen). The rabbit antibody was detected with the goat anti-rabbit IgG-HRP (Tago). The color reaction was developed with 3, 3′, 5, 5′tetramethylbenzidine substrate and the OD was measured at 450 nM. The amount of IL-6 was calculated compared to a standard curve of the recombinant human IL-6 (Genzyme).

Results and Discussion

Reactivity with Monoclonal Antibodies

The mutants generally fell into two groups (Table 1). The DDED, PQPD, DDED/PQPD and AVEVE deletions all had poor reactivity with 28C5, MY-4 and 60bca, UCHM-1 and MO-2, but good reactivity with 18E12 and 63D3. 28C5, MY-4 and 60bca have been shown to inhibit CD14 binding to LPS (10,26). 63D3 and 18E12 likely bind to epitopes C-terminal to glycine 152, since they don't react with the 152 amino acid CD14 truncation (28). The DPRQY deletion was reactive with all mAbs tested, except for MEM-18. These patterns of reactivity are what would be predicted from previous studies with soluble (31) and membrane CD14 (26,28). The results are depicted in the following table, where an OD of >90% of the OD wild-type CD14 was considered ++++, 76-90% assigned +++, 51-75% assigned ++, and 11-50% assigned +.

| | | | | | | | | | POLY- |
|---|---|---|---|---|---|---|---|---|---|
| DELETION | 28C5 | MY4 | 60bca | MEM-18 | UCHM-1 | MO-2 | 18E12 | 63D3 | CLONAL |
| DDED | 0 | 0 | 0 | + + + | + | 0 | + + + + | + + + + | + + + + |
| PQPD | + | 0 | + | + + + | + | 0 | + + + | + + + | + + + + |
| DDED/PQPD | 0 | 0 | 0 | + + + | 0 | 0 | + + + + | + + + + | + + + + |
| AVEVE | 0 | 0 | 0 | + + | 0 | 0 | + + | + + | + + + |
| DPRQY | + + + + | + + + + | + + + + | 0 | + + + + | + + | + + + + | + + + + | + + + + |

LPS Binding

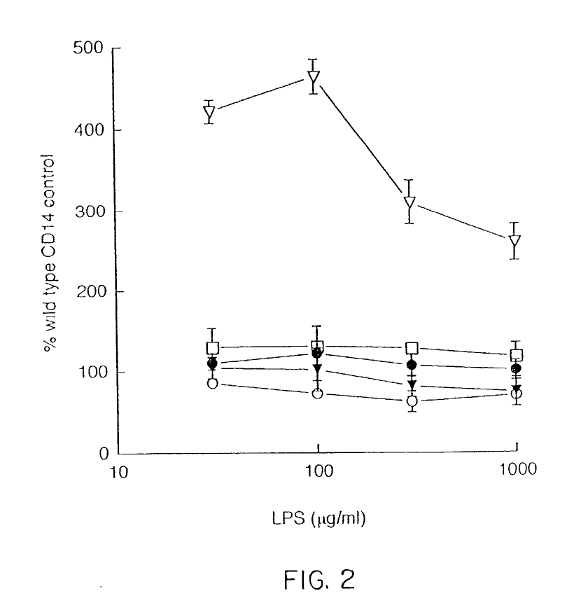

CD14 binding of LPS was measured in three different ways: ELISA, binding of FITC-LPS, and sucrose density gradient analysis of complexes. The results of a typical ELISA assay are shown in FIG. 2. Four of the five deletion mutants bound LPS approximately as well as wild-type CD14. The DDED/PQPD deletion mutant bound a maximum of 4.5 times more LPS than did wild-type CD14. Results are described in the table below, according to the following: LPS binding: −, undetectable binding, +, <10% of wild-type; ++++, approximately the same as wild-type; >++++, more than 10 fold better than wild type. LPS activation: −, undetectable activation; +/−activation only at high concentrations of CD14; ++30-50% of wild-type; +++, >50% of wild type.

| | |
|---|---|
| LPS BINDING | LPS ACTIVATION |

| Deletion | mCD14 | sCD14 | mCD14 | sCD14 |
|---|---|---|---|---|
| DDED | − | + + + + | + + | − |
| PQPD | − | + + + + | + + | − |
| DDED/PQPD | − | > + + + + | − | − |
| AVEVE | − | + + + + | − | − |
| DPRQY | − | + | + + + | − |

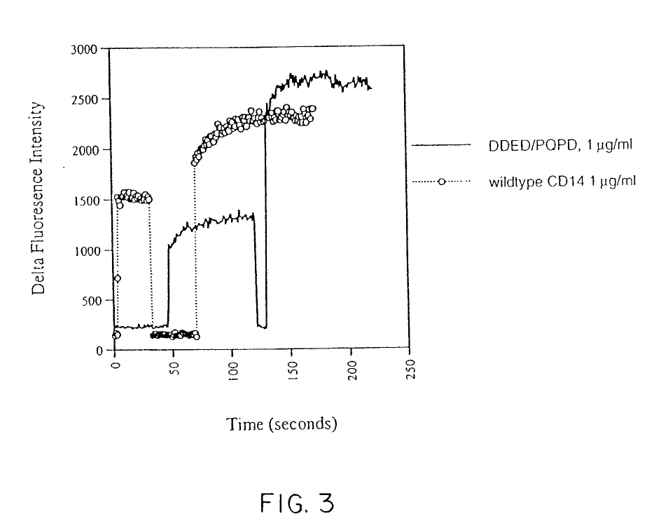

We also examined the soluble CD14 deletion mutants in the FITC-LPS binding assay. A representative tracing of wild-type CD14 binding FITC-LPS is shown (FIG. 3). Using our preparations of sCD14, there appears to be no need for LPS binding protein to observe the increase in fluorescent signal that is associated with CD14 binding of LPS. FIG. 3 shows a tracing of the fluorescence seen when wild-type CD14 or the DDED/PQPD double mutant is added to FITC-LPS. 1 mg/ml of the PDED/PQPD deletion mutant caused a larger increase in fluorescence than 1 mg/ml wild-type CD14. To evaluate the effect of the 6His tag on LPS binding in this assay, we compared wild-type CD14 and the DDED/PQPD deletion mutant, with and without the 6His tag. The binding curves were identical regardless of the presence of the 6His tag (data not shown). We obtained estimates of apparent K_{D} of all the mutants by measuring the increase in fluorescence intensity a function of concentration of CD14 (Table 2). The estimated apparent K_{D} of the CD14/LPS interaction for the deletion mutant varied from 2×10⁻⁹ (DDED/PQPD) to <10⁶ (DPRQY). Although the fluorescence assay allows us to measure affinity of sCD14 for LPS, results obtained with LBP and bactericidal/permeability increasing protein show that the assay does not report LPS aggregation state (34).

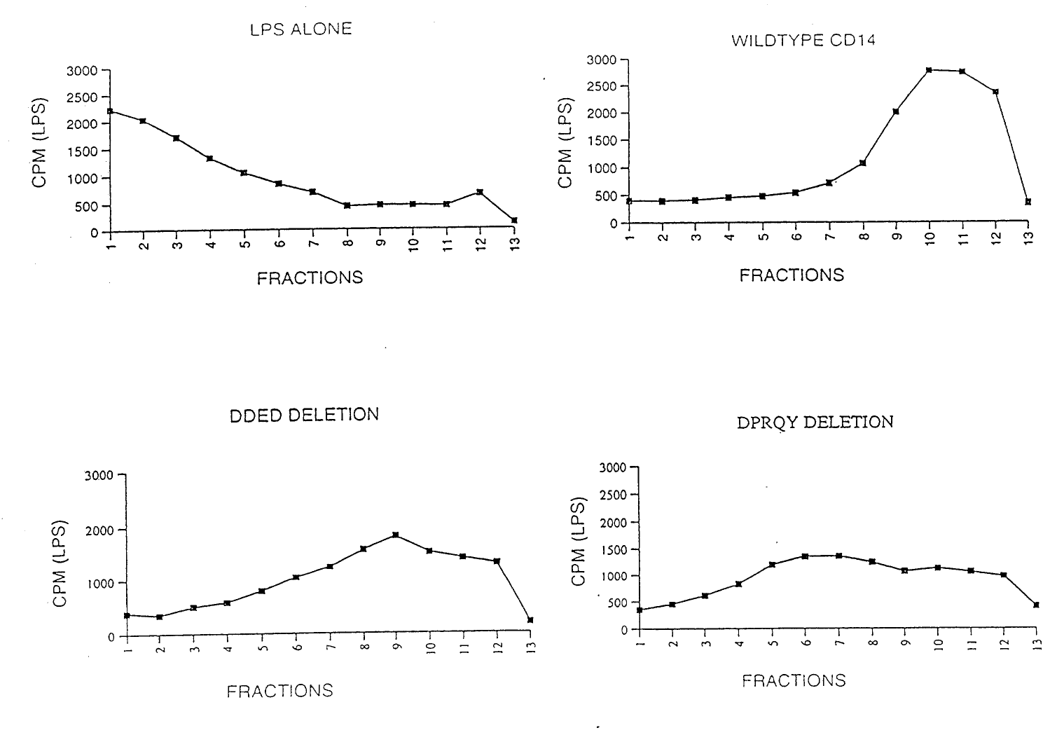

The functional consequences of LPS binding by different proteins can be very different. For example, both LBP and bactericidal/permeability increasing protein bind FITC-LPS with an increase in fluorescence similar to those seen in FIG. 3, but LPS complexed with bactericidal/permeability increasing protein form aggregates and LPS complexed with LBP is dispersed (34). To address the aggregation state, sucrose density gradient experiments were done to evaluate the sedimentation velocity of LPS complexes with mutant CD14. FIG. 4 shows the results. As expected, LPS alone migrates to the bottom of the tube indicating that it is aggregated (FIG. 4A). Wild-type CD14: LPS complexes are primarily at the top of the gradient indicating that the complexes are relatively small (FIG. 4B). We interpret these data as we have in the past (32, 34), that sCD14m has dissociated the large LPS aggregates by forming containing one or a few LPS molecules per sCD14. The complexes of LPS with the PQPD, DDED/PQPD and AVEVE deletion mutants (data not shown) all resembled LPS: wild-type CD14 complexes (FIG. 4B). LPS complexed with the DDED deletion (FIG. 4C) or the DPRQY deletion (FIG. 4D) were more widely distributed in the gradient, but generally had a higher sedimentation velocity than wild-type CD14: LPS complexes. Overall, these studies showed that sCD14 mutants with high affinity for LPS (as measured by the fluorescence assay) also formed low molecular weight LPS:CD14 complexes.

Biological Activity of the Deletion Mutants

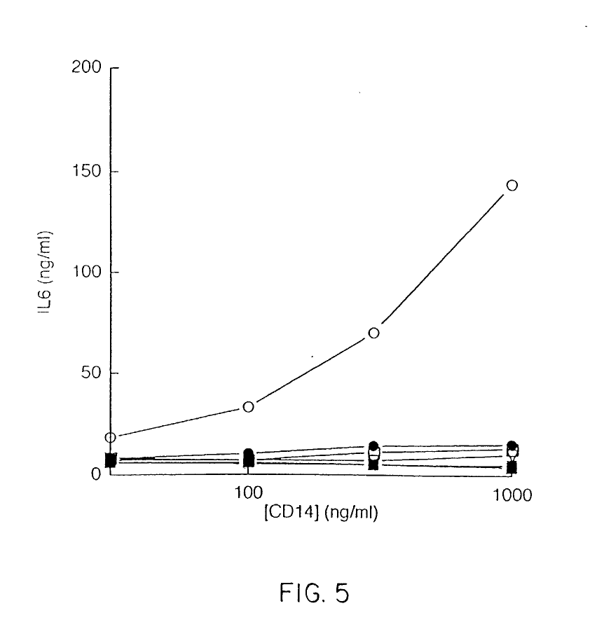

The biological activity of the various forms of CD14 were determined using a U373 epithelial cell assay for sCD14 activity. Soluble wild-type or mutant CD14 alone did not activate these cells. The concentration of Re LPS was 10 ng/ml, and the concentration of CD14 or deletion mutant varied from 0 to 1 mg/ml. Over this concentration range, there was very little, if any, activation of U373 cells by the CD14 mutants as assessed by IL-6 release (FIG. 5). Once again, the DDED/PQPD deletion mutant behaved the same with or without the 6His tag (data not shown). Similar results with the CD 14 were seen with E. coli O111: B4 LPS stimulating U373 cells to produce IL-6 and Re LPS stimulating SW620 cells to produce IL-8 (data not shown). At higher concentrations, some of the mutants did stimulate cellular responses (see below).

### Example 5

### Use of the Mutants as Competitive Inhibitors

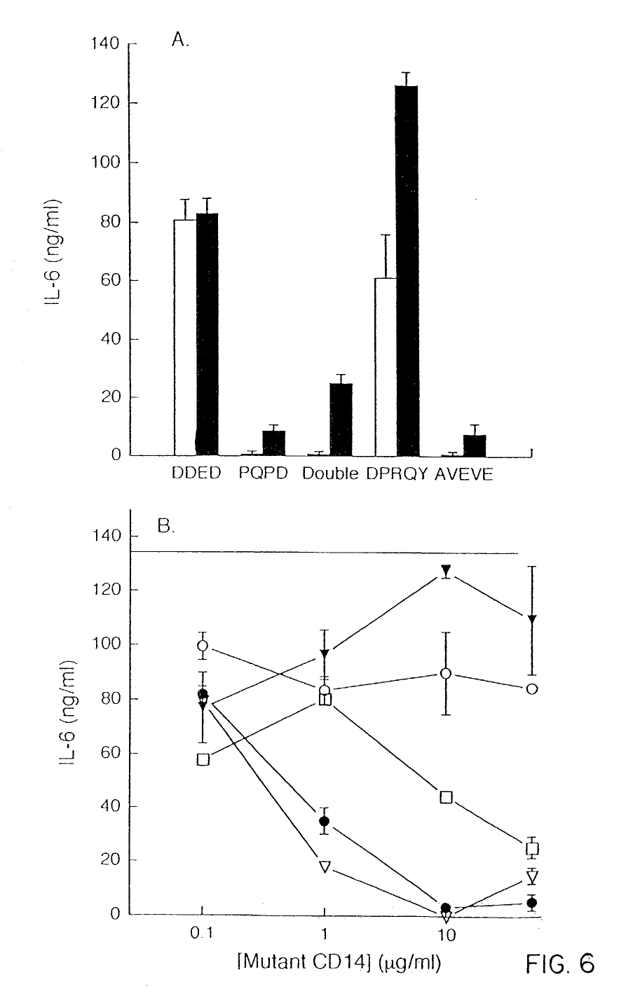

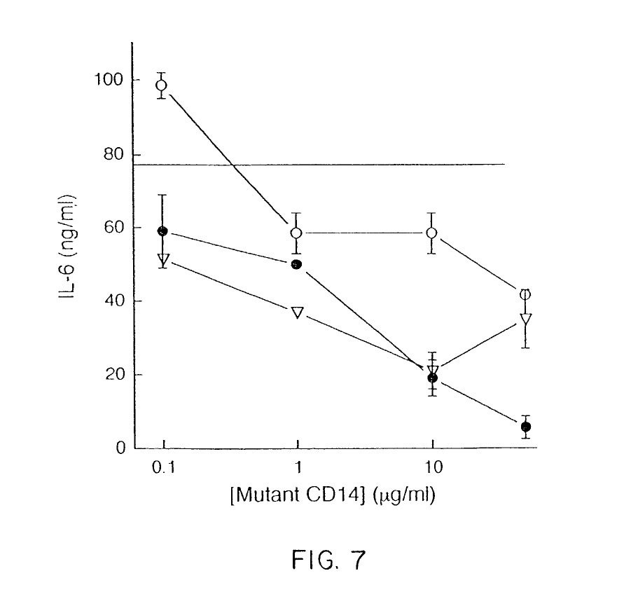

Since many of the CD14 mutants bound LPS but did not activate cells, we asked whether they would compete with wild-type CD14 and inhibit sCD14-dependent cell activation. A typical experiment is shown in FIG. 6. Panel A shows the response to 10 ng/ml Re595 LPS in the presence of 10 or 50 mg/ml mutant CD14. High concentrations of the DDED and the DPRQY mutants activated U373 in the presence of LPS. In the experiment shown in Panel B, the concentration of Re LPS was 10 ng/ml and the concentration of wild type sCD14 was 200 ng/ml. The concentration of mutant CD14 was varied from 0.1 to 50 mg/ml. The PQPD, DDED/PQPD and AVEVE mutants inhibited activation of U373 cells by wild type CD14, in a concentration dependent fashion. To ensure that this phenomenon was not related to the 6His tag, the DDED/PQPD mutant with the 6His tag removed was tested, and was found to inhibit LPS activation as well as the tagged molecule. In other experiments with Re LPS, E. coli D31 m4 LPS and E. coli O111: B4 LPS, the DDED/PQPD mutant was consistently inhibitory at concentrations of 1 mg/ml or above.

To determine whether or not the inhibition seen with mutant CD14 was specific for responses stimulated by LPS, U373 cells were stimulated with 1 ng/ml IL-1 b in the presence or absence of wild-type or mutant CD14. In all instances, there was no inhibition of IL-1 b stimulation of IL-6 by the DDED, DDED/PQPD or the AVEVE CD14 deletion mutants (data not shown).

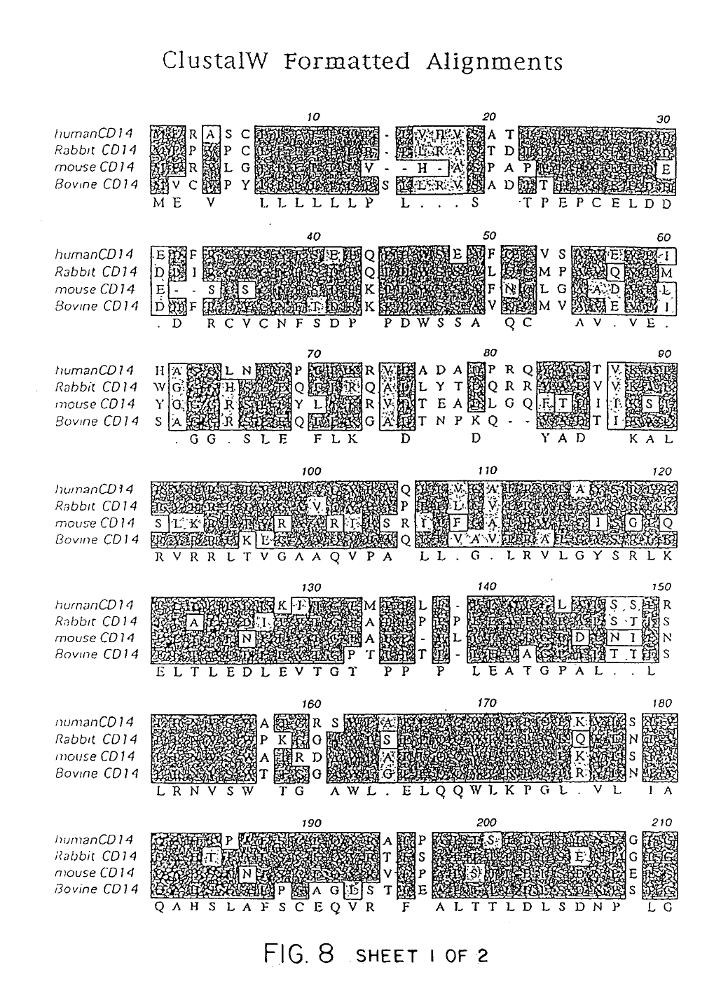

These studies were done with wild-type and mutant CD14 in the absence of other plasma proteins which might modulate the interaction of LPS with CD14. In an effort to more closely mimic the in vivo situation, other experiments were done using diluted normal human serum as a source of wild-type CD14. FIG. 7 shows the results. In this experiment, the PQPD and AVEVE mutants partially inhibited wild-type CD14 LPS receptor function. In contrast, at concentrations of 10 to 50 mg/ml, the DDED/PQPD deletion mutants almost completely inhibited LPS-induced IL-6 production by U373 cells in the presence of serum.

Discussion

The objective of this study was to compare membrane and soluble forms of CD14 mutants directly in several different assay systems. They included monoclonal antibody reactivity, LPS binding, and LPS activation of cells. The reactivity with mAbs of the sCD14 mutants was almost identical to the pattern of reactivity seen with the membrane form of the protein. There appear to be two classes of epitopes within the N-terminal half of the protein; the 28C5, MY-4, 60bca, MO-2 group (26,28) and the MEM-18, CHRIS 6 group (31). Although both of these mAb inhibit CD14 binding of LPS and activation of cells, they clearly react with different epitopes by mutational analysis. The epitopes recognized by 63D3 and 18E12 appear to be the C-terminal half of the protein since they do not react with the N-terminal 1-152 amino acid truncation mutant, in either membrane or soluble form (28,35).

The three LPS binding assays used gave different perspectives on the ability of the CD14 mutants to bind LPS. In the ELISA assays, all the mutants, including the DPRQY deletion bound LPS as well as wild-type. In previous work, a D57-64 deletion mutant (a DADPRQYA deletion) was found not to bind LPS in a native gel binding assay (31). We presume that the deletion of three additional amino acids is responsible for the lack of LPS binding activity of that mutant in contrast to our results with the DPRQY deletion. The ELISA assay revealed that the DDED/PQPD double deletion bound more LPS than wild-type CD14, and suggested that all of the other mutants bound LPS normally. The FITC-LPS fluorescence assay was the most quantitative LPS binding assay (32). By varying the protein concentration, and measuring the increase in fluorescent signal, we could estimate apparent K_{D}. The apparent K_{D} observed for the wild-type CD14 binding to LPS was essentially identical to previous estimates for sCD14 (32), and very similar to the apparent KD estimated for membrane CD14 binding of ³H-LPS (36). The DDED, PQPD and AVEVE deletions all bound slightly better than wild-type CD14. Unexpectedly, the DDED/PQPD double deletion bound LPS with almost 40 fold higher apparent affinity than wild-type CD14. As expected from a previous study (31), the DPRQY bound LPS very poorly in the fluorescence assay. The findings are summarized in the table below.

| CD14 | K_{D} (M⁻¹) |
|---|---|
| Wild-type | 7.4 × 10⁻⁸ |
| DDED deletion | 1.7 × 10⁻⁸ |
| PQPD deletion | 1.7 × 10⁻⁸ |
| DDED/PQPD deletion | 2 × 10⁻⁹ |
| AVEVE deletion | 3.3 × 10⁻⁸ |
| DPRQY deletion | <10⁻⁶ |

Sucrose density gradient assays gave additional information about the nature of the complexes formed. All mutants formed complexes with ³H-LPS that had a lower sedimentation velocity than LPS alone, which suggests that the LPS is disaggregated when bound to the CD14 mutants. Even the DPRQY mutant, which bound LPS poorly in the fluorescence assay, formed some complexes.

The preparations of CD14 we used bound LPS rapidly in the absence of LBP. We cultivated our baculovirus-infected High 5 insect cells in Excel 405 serum-free medium. In experiments to be reported elsewhere, we found that CD14 derived from insect cells cultured in this medium containing 5% fetal calf serum required LBP for efficient binding of LPS in the fluorescent LPS binding assay, in contrast to CD14 prepared from insect cells cultured in serum-free medium. We are currently comparing sCD14 prepared in serum and in the absence of serum to try to determine the basis of the need for LBP.

Physiologic concentrations of all but one of the sCD14 mutants we have reported here are unable to activate U373 and SW620 cells in the presence of LPS, despite their ability to bind LPS. We expected that the DDED deletion would lack activity because of previous reports that this region was critical for sCD14 LPS receptor function (37). The DPRQY region has previously been identified as critical for LPS binding activity of sCD14 (31). Darveau reported that a charge reversal point mutation of E47®R47 (which is located between the PQPD and the AVEVE regions) selectively effected the binding of Porphyromonas gingivalis LPS (30).

The E47®R47 mutant was also a less biologically active receptor for P. gingivalis LPS in an endothelial cell activation assay (30).

The observation that all but one of our sCD14 mutants were biologically inactive LPS receptors was unexpected. When these same deletions were evaluated as cell membrane LPS receptors on CHO cells and 70Z/3 cells, we found that receptor function was modestly impaired in the DDED and PQPD deletions (26,28) (Table 3). The DDED/PQPD double deletion and the AVEVE deletion were almost inactive as membrane LPS receptors (26). The DPRQY deletion was minimally impaired in receptor function (26). All the deletions were incapable of binding detectable amounts of LPS as membrane receptors in several different types of assay (26). Clearly, the critical regions in CD14 for LPS receptor function are different in the membrane and soluble forms of the receptor. It is widely believed that membrane CD14 is only part of a receptor complex and that sCD14:LPS complexes must also bind to a signal transducing receptor. The discrepancy between critical regions of sCD 14 and mCD 14 suggests that these two forms of CD 14 may be interacting with different signal transducing receptors.

The ability of some of these mutants to competitively inhibit sCD 14 and membrane CD14 receptor function makes sense because of the high affinity binding of LPS without LPS receptor activity. The DDED/PQPD double deletion is particularly striking in this regard. This mutant is capable of complete inhibition of sCD14 receptor function in a concentration dependent manner. The inhibition is specific for LPS, since responses to IL-1 b are not effected. The DDED/PQPD double deletion can also inhibit sCD14 receptor function in human serum, indicating that it is capable of LPS binding in the presence of other LPS binding proteins. The ability of these sCD14 mutants to inhibit LPS activation of cells in vitro suggests that they may also be able to inhibit LPS toxicity in vivo.

This likelihood is bolstered by favorable in vivo results recently achieved in a murine model system. (See, e.g., U.S. Pat. No. 5,804,189 herein incorporated by reference). There, acute sepsis was induced in mice by injection of otherwise lethal doses of microbe, and quickly reversed by the administration of solid human CD-14 that differed only in glycosylation pattern. In other in vitro studies, small peptide mimetics possessing the LPS cognate binding region (decoupled) were effective to reverse CD-14 mediated symptoms of sepsis. (See U.S. Pat. No. 5,766,593 herein incorporated by reference) Presumably, the mimetics used in the above studies functioned as competitive inhibitors to reverse the otherwise harmful effects of systemic infection.

The foregoing studies, however, did not teach modifications of the nature and merit taught by Applicants. Applicants teach an exceptionally potent CD-14 inhibitor that, because of its potency and substantial full-length identity with its native counterpart, provides advantages of reduced immunogenicity and other side-effects when offered as a therapeutic.

The following references accompany the foregoing discussion, and are herein incorporated by reference in the numerical order in which they appear in the document.

1. Bone, R. C. (1991) Ann. Intern. Med. 115, 457-469

2. Parrillo, J. E., Parker, M. M., Natanson, C., Suffredini, A. F., Danner, R. L., Cunnion, R. E., and Ognibene, F. P. (1990) Ann. Intern. Med. 113, 227-242

3. Ulevitch, R. J. (1993) Adv. Immunol. 53, 267-289

4. Raetz, C. R. H., Ulevitch, R. J., Wright, S. D., Sibley, C. H., Ding, A., and Nathan, C. F. (1991) FASEB J. 5, 2652-2660

5. Frey, E. A., Miller, D. S., Jahr, T. G., Sundan, A., Bazil, V., Espevik, T., Finlay, B. B., and Wright, S. D. (1992) J. Exp. Med. 176, 1665-1671

6. Pugin, J., Schurer-Maly, C. C., Leturcq, D., Moriarty, A., Ulevitch, R. J., and Tobias, P. S. (1993) Proc. Natl. Acad. Sci. USA 90, 2744-2748

7. Pugin, J., Ulevitch, R. J., and Tobias, P. S. (1993) J. Exp. Med. 178, 2193-2200

8. Moore, K. L., Andreoli, S. P., Esmon, N. L., and Bang, N. U. (1987) J. Clin. Invest. 79, 124-130

9. Kilbourn, R. G., Gross, S. S., Jubran, A., Adams J., Griffith, O. W., Levi, R., and Lodato, R. F. (1990) Proc. Nat. Acad. Sci. USA 87, 3629-3632

10. Wright, S. D., Ramos, R. A., Tobias, P. S., Ulevitch, R. J., and Mathison, J. C. (1990) Science 249, 1431-1433

11. Lee, J. D., Kato, K., Tobias, P. S., Kirkland, T. N., and Ulevitch, R. J. (1992) J. Exp. Med. 175, 1697-170512. Gupta, D., Kirkland, T. N., Virlyakosol, S., and Dzarski, R. (1996) J. Biol. Chem. 271, 23310-23316

13. Haziot, A., Chen, S., Ferrero, E., Low, M. G., Silber, R., and Goyert, S. M. (1988) J. Immunol. 141, 547-552

14. Haziot, A., Ferrero, E., Kontgen, F., Hijiya, N., Yamamoto, S., Silver, J., Stewart, C., Goyert, S. (1996) Immun. 4, 407-414

15. Kirikae, T., Schade, F. U., Kirikae, F., Rietschel, E. T., and Morrison, D. C. (1993) J. Immunol. 151, 2742-2752

16. Dziarski, R., Tapping, R. I., and Tobias, P. S. (1998) J. Biol. Chem. 273, 8680-8690

17. Peterson, P. K., Gekker, G., Hu, S., Sheng, W. S., Anderson, W. R., Ulevitch, R. J., Tobias, P. S., Gustafson, K. V., Molitor, T. W., and Chao, C. C. (1995) Infect. Immun. 63, 1598-1602

18. Kusunoki, T., Hailman, E., Juan, T. S., Lichenstein, H. S., and Wright, S. D. (1995) J. Exp. Med. 182, 1673-1682

19. Espevik, T., Otterlel, M., Skjak-Braek, G., Ryan, L., Wright, S. D., and Sundan, A. (1993) Eur. J. Immunol. 23, 255-261

20. Sellati, T. J., Bouls, D. A., Kitchens, R. L., Darveau, R. P., Pugin, J., Ulevitch, R. J., Gangloff, S. C., Goyert, S. M., Norgard, M. V., and Radolf, J. D. (1998) J. Immunol. 160, 5455-5464

21. Hogan, L. H., Josuai, S., and Klein, B. S. (1995) J. Biol. Chem. 270, 30725-30732

22. Devitt, A., Moffatt, O. D., Raykundalia, C., Capra, J. D., Simmons, D. L., and Gregory, C. D. (1998) Nature 392, 505-509

23. Wurfel, M. M. and Wright, S. D. (1997) J. Immunol. 158, 3925-3934

24. Wang, P., Kitchens, R., and Munford, R. (1998) J Biolog Chem 0.273, 24309-24313.

25. Janeway, C. A. (1992) Immunol. Today 13, 11-16

26. Viriyakosol, S., and Kirkland, T. N. (1995) J. Biol. Chem. 270, 361

27. Virlyakosol, S., and Kirkland, T. (1995) Clin. Infect. Dis. 21(suppl.2), S190-195

28. Viriyakosol, S., and Kirkland, T. N. (1996) Infect. Immun. 64, 653-656

29. Ulevitch, R., Johnston, A., and Weinstein, D. (1981) J. Clin. Invest. 67, 827-837

30. Shapiro, R. A., Cunninghan, M. D., Ratcliffe, K., Seachord, C., Blake, J., Bajorath, J., Aruffo, A., and Darveau, R. P. (1997) Infect. Immun. 65, 293-297

31. Juan, T. S. C., Hailman, E., Kelley, M. J., Busse, L. A., Davy, E., Empig, C. J., Narhi, L. 0., Wright, S. D., and Lichenstein, H. S. (1995) J. Biol. Chem. 270, 5219-5224

32. Tobias, P. S., Soldau, K., Gegner, J. A., Mintz, D., and Ulevitch, R. J. (1995) J. Biol. Chem 270, 10482-10488

33. Desch, C. E., Kovach, N. L., Present, W., Broyles, C., and Harlan, J. (1989) Lymphokine Research 8, 141-146

34. Tobias, P. S., Soldau, K., Iovine, N. M., Elsbach, P., and Weiss, J. (1997) J. Biol. Chem. 272, 18682-1868535. Juan, T. S. C., Kelley, M. J., Johnson, D. A., Busse, L. A., Hailman, E., Wright, S. D., and Lichenstein, H. S. (1995) J. Biol. Chem. 270, 1382-1387

36. Kirkland, T. N., Finley, F., Leturcq, D., Moriarty, A., Lee, J. D., Ulevitch, R. J., and Tobias, P. S. (1993) J. Biol. Chem. 268, 24814-24823

37. Juan, T. S. C., Hailman, E., Kelley, M. J., Wright, S. D., and Lichenstein, H S. (1995) J. Biol. Chem. 270, 17237-17242

Although preferred embodiments of the invention have been described in the examples and detailed prophetic embodiments also given, it will be understood by those skilled in the field that modifications may be made to the disclosed embodiment without departing from the scope of the invention, which is further defined in the appended claims, below.

We claim:

1. A modified mammalian CD14 receptor comprising:
a) a first domain capable of binding a membrane component from a foreign body with an enhanced affinity relative to the same domain in an unmodified mammalian CD14 receptor, and
b) a second domain known to be associated with transmission of a cellular signal, said second domain altered to reduce the transmission of said signal upon the binding of said membrane component by said first domain.

2. The modified mammalian CD14 receptor of claim 1 wherein said receptor is substantially full-length relative to said unmodified receptor.

3. The modified mammalian CD14 receptor of claim 1 wherein said first domain binds component from a foreign body selected from the group consisting of gram negative bacteria and gram positive bacteria, and wherein said foreign body is correlatable with the symptoms of sepsis or septic shock in a mammalian host.

4. The modified mammalian CD14 receptor of claim 1 wherein said foreign body is a gram negative bacterium that produces LPS molecules that are capable of transmitting cellular signals upon binding of an unmodified CD14 receptor in a mammalian host.

5. The modified mammalian CD14 receptor of claim 1 wherein said enhanced binding affinity of said first domain is a consequence of the alteration of said second domain.

6. The modified mammalian CD14 receptor of claim 1 wherein said first domain is separated from said second domain by about 50 amino acid residues when positioned in a primary array.

7. The modified mammalian CD14 receptor of claim 1 wherein said altered second domain comprises an otherwise functional domain to which have been made specific deletions of amino acid residues constituting a sub-part thereof.

8. The modified mammalian CD14 receptor of claim 1 wherein said enhanced affinity of said first domain results from changes introduced at a remote site thereto on the receptor.

9. The modified mammalian CD14 receptor of claim 1 wherein said enahnced affinity of said first domain is at least an order of magnitude higher than that exhibited by the unmodified receptor.

10. The modified mammalian CD14 receptor of claim 1 wherein said first domain is essentially unchanged from a corresponding domain in said unmodified mammalian CD14 receptor, and wherein said enhanced affinity is introduced by alterations made at a remote site thereto.

11. The modified mammalian CD14 receptor of claim 1 wherein said receptor comprises an amino acid sequence selected from the group consisting of human and murine.

12. The modified mammalian CD14 receptor of claim 11 wherein said second domain comprises the deletion of amino acid sequences corresponding to residues 9-12 and 22-25 of Seq. ID No: 1.

13. The modified mammalian CD14 receptor of claim 1 wherein said receptor is included as part of a diagnostic kit.

14. The modified mammalian CD14 receptor of claim 1 wherein said receptor is recombinantly grown in an insect cell system that makes use of a baculovirus expression vector.

15. The modified mammalian CD14 receptor of claim 1 wherein said receptor is included as part of a pharmaceutical composition.

16. The pharmaceutical composition of claim 15 wherein said composition is used for the treatment of symptoms associated with sepsis or septic shock.


SEQUENCE LISTING (5 sequences)

SEQ ID NO: 1 - protein, 339 aa, Homo sapiens
  MISC_FEATURE (326)..(339): residues are contributed by linker and vector
TEPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSYEQVRAFPALTSLDLSDNPGLGERGLMAALFPHKPFPAIQNLALPRNTGMETPTGVCAALAAAGVQPHSLDLSHNSLRATVNPSAPRCMWSSLANSLNLSFAGLEQVPKGLPAKLRVLDLSCNRLNRAPQPDELPEVDNLTLDGNPFLVPGTALPHEGEFHHHHHHKLVEKY

SEQ ID NO: 2 - DNA, 31 bp, Artificial Sequence
  CD14 PCR primer sequence fused to restriction site sequence
cggatccatggagcgcgcgcgtcctgcttgt

SEQ ID NO: 3 - DNA, 30 bp, Artificial Sequence
  CD14 PCR primer sequence fused to restriction site sequence
gggtcagtgctgcaacattttgctgccggt

SEQ ID NO: 4 - DNA, 24 bp, Artificial Sequence
  CD14 PCR primer sequence fused to restriction site sequence
aattccatcaccatcaccatcaca

SEQ ID NO: 5 - DNA, 24 bp, Artificial Sequence
  CD14 PCR primer sequence fused to a 6x His tag
agcttgtgatggtgatggtgatgg